(19) United States
(12) Patent Application Publication
(10) Pub. No.: US 2015/0231081 A1
(43) Pub. Date: Aug. 20, 2015

(54) DELAYED RELEASE POSACONAZOLE TABLETS

(71) Applicant: CADILA HEALTHCARE LIMITED, Ahmedabad (IN)

(72) Inventors: Sushrut Krishnaji KULKARNI, Ahmedabad (IN); Pavak Rajnikant MEHTA, Ahmedabad (IN); Ritesh KAPOOR, Ahmedabad (IN); Rajesh Kumar Bharat MAHESHWARI, Ahmedabad (IN)

(73) Assignee: CADILA HEALTHCARE LIMITED, Ahmedabad (IN)

(21) Appl. No.: 14/626,207

(22) Filed: Feb. 19, 2015

(30) Foreign Application Priority Data
Feb. 20, 2014 (IN) 589/MUM/2014

(51) Int. Cl. A61K 9/20; A61K 9/28; A61K 31/496

(52) U.S. Cl. CPC A61K 9/2027; A61K 31/496; A61K 9/2095; A61K 9/2893; A61K 9/28

(57) ABSTRACT

The present invention relates to a delayed release composition comprising Posaconazole dissolved or molecularly dispersed in a polymer other than a hydroxypropyl methylcellulose derived polymer; wherein the composition is prepared by hot melt extruding an admixture of Posaconazole and the polymer. The present invention also provides a process of preparing said composition.

### FIELD OF INVENTION

The present invention relates to a delayed release pharmaceutical composition comprising Posaconazole, and a polymer other than a hydroxypropyl methylcellulose derived polymer and one or more pharmaceutically acceptable excipients, wherein the composition is prepared by hot melt extrusion process. It also relates to method of preparing such compositions and using those compositions in the prevention and/or treatment of a fungal infection or related disease.

### BACKGROUND OF INVENTION

Posaconazole is designated chemically as 4-[4-[4-[4-[[(3R,5R)-5-(2,4-difluorophenyl)tetrahydro-5-(1H-1,2,4-triazol-1-ylmethyl)-3-furanyl]methoxy]phenyl]-1-piperazinyl]phenyl]-2-[(1S,2S)-1-ethyl-2-hydroxypropyl]-2,4-dihydro-3H-1,2,4-triazol-3-one and has the following structural formula:

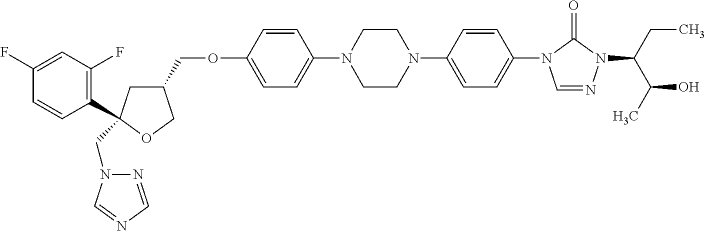 [SMILES: CC[C@@H]([C@H](C)O)n1ncn(-c2ccc(N3CCN(c4ccc(OC[C@@H]5CO[C@@](Cn6cncn6)(c6ccc(F)cc6F)C5)cc4)CC3)cc2)c1=O]

Posaconazole is an azole compound with antifungal properties. A pharmaceutical composition comprising a suspension of crystalline form (available commercially as Noxafil®) and a method for preparing the same are described in published U.S. Patent Application No. 2003/0055067.

A suspension containing Posaconazole in crystalline form (40 mg/mL) has been approved inter alia in the U.S. and European Union as Noxafil® for oral administration in the treatment of invasive fungal infections, for example, the treatment of oropharangyl candidiasis, including infections which are resistant to treatment by other azole antifungals, and as a prophylatic treatment to prevent fungal infections in patients who are at high risk of developing these infections due to being severely immunocompromised, such as hematopoietic stem cell transplant (HSCT) recipients with graft-versus-host disease (GVHD) or those with hematologic malignancies with prolonged neutropenia from chemotherapy. Noxafil® is indicated for oral administration with food, preferably a high-fat meal (or in severely neutropenic patients unable to tolerate food intake, after administration of a nutritional supplement) in order to assure attainment of adequate plasma concentrations of Posaconazole. As reported in the PDR, administration of Noxafil® to a patient with a high-fat meal exhibits an increase in drug plasma concentration which is four times over what is observed after administration of an equivalent amount of Noxafil® to a fasting patient (also termed herein, “Fasted Conditions”), and exhibits 3 times increase in plasma concentration when administered to patients along with a nutritional supplement over what is observed after administration of Noxafil® to a fasted patient.

The provision of a solid composition comprising Posaconazole suitable for preparing a solid dosage form for oral administration has heretofore been hampered by the poor solubility and weak basicity of the Posaconazole free-base compound. Posaconazole is soluble at lower pH. For example, Posaconazole free base has a solubility of approximately 0.8 mg/mL in stomach pH. However, when Posaconazole dissolved in the stomach fluids reaches the environment of the intestines (typically less acidic than about pH 6.4) a substantial amount of the dissolved Posaconazole precipitates, hindering absorption in the intestines. It has been determined that in environments where the pH is about pH 6.4 or more basic the solubility of Posaconazole free base is less than about 1 microgram/mL.

U.S. Pat. No. 7,235,260 (the '260 patent), describes glycogen phosphorylase inhibitors in hydroxypropylmethylcel lulose and hydroxypropylmethylcellulose-derivative polymers. The compositions described in the '260 patent are prepared by spray-drying a solution containing a phosphorylase inhibitor and hydroxypropyl methylcellulose acetate succinate (HPMC-AS) dissolved in a common solvent. U.S. Pat. No. 6,881,745 (the '745 patent), generally describes compositions comprising an azole antifungal compound and a polymer. The compositions described are prepared by dissolving the azole compound and polymer in a common solvent, for example, methylene chloride, chloroform, ethanol, methanol, isopropanol, ethyl acetate, acetone, or mixtures thereof, and forming a solid granular composition by spray-drying the solution using conventional spray-drying equipment. An example of an azole-containing composition described in the '745 patent is itraconazole with a hydroxypropyl methylcel lulose-phthalate (HPMC-phthalate) polymer derivative prepared by spray-drying a solution containing the active pharmaceutical ingredient (API) and the polymer.

U.S. Patent publication No. 2011/0123627 describes compositions comprising Posaconazole dissolved or molecularly dispersed in a hydroxypropylmethylcellulose-derivative polymer (HPMC-derivative polymer). The invention relates to selected grades of HPMC-AS polymer for use in compositions, which leads to little or no decomposition of the Posaconazole used in the composition.

U.S. Patent publication No. 2007/0281011 describes nanoparticulate Posaconazole compositions comprising a surface stabilizer adsorbed on the surface of the Posaconazole particles. The composition process requires milling Posaconazole particles in a liquid dispersion medium in which Posaconazole is poorly soluble, followed by applying mechanical means in the presence of grinding media to reduce the particle size of the Posaconazole to the desired effective average particle size.

It has been found that delayed release Posaconazole compositions can also be prepared using polymers other than a hydroxypropyl methylcellulose derived polymer using hot melt extrusion technique. The instant invention addresses this unmet need by providing alternate compositions of Posaconazole.

### SUMMARY OF THE INVENTION

In a general aspect, there is provided a delayed release composition comprising Posaconazole dissolved or molecularly dispersed in a polymer other than a hydroxypropyl methylcellulose derived polymer; wherein the composition is prepared by hot melt extruding an admixture of Posaconazole and the polymer.

In another general aspect, there is provided a delayed release composition; wherein the composition comprises Posaconazole up to 30% by weight of the composition.

In another general aspect, there is provided a delayed release composition; wherein the composition comprises from about 100 mg to about 400 mg Posaconazole.

In another general aspect, there is provided a composition wherein the composition does not give significantly different absorption levels when administered under fed conditions as compared to fasting conditions.

In another general aspect, there is provided a composition wherein the composition comprises the polymer from about 5% to about 20% by weight of the composition.

Embodiments of the pharmaceutical composition may include one or more of the following features. For example, the pharmaceutical composition may further include one or more pharmaceutically acceptable excipients. The pharmaceutically acceptable excipients may include one or more fillers, disintegrants, binders, wetting agents, lubricants, glidants and the like.

In another general aspect, there is provided a delayed release composition, wherein the composition is prepared by process comprising the steps of:

a) blending Posaconazole with the polymer other than a hydroxypropyl methylcellulose derived polymer and one or more pharmaceutically acceptable excipients to form an admixture,
b) heating the admixture to form a melt,
c) extruding the melt into a shaped mass followed by or simultaneously cooling the extrudate,
d) sizing the extrudate through a co-mill to achieve desired granules,
e) blending the granules with one or more pharmaceutically acceptable excipients followed by lubrication,
f) compressing the lubricated blend into tablets, and optionally;
g) coating the tablets.

In another general aspect, there is provided a method for preventing and/or treating a fungal infection or related disease comprising administering the Posaconazole composition of the invention.

Embodiments of the pharmaceutical composition may include one or more of the following features. For example, the pharmaceutical composition may further include one or more pharmaceutically acceptable excipients. The pharmaceutically acceptable excipients may include one or more fillers, disintegrants, binders, wetting agents, lubricants, glidants and the like.

The details of one or more embodiments of the invention are set forth in the description below. Other features of the invention will be apparent from the description.

### DETAILED DESCRIPTION OF THE INVENTION

The present invention provides a delayed release composition of Posaconazole for oral administration; wherein the composition comprises Posaconazole dissolved or molecularly dispersed in a polymer other than a hydroxypropyl methylcellulose derived polymer.

It has now surprisingly been found that a Posaconazole composition can be prepared by hot melt extrusion process with the use of polymers other than a hydroxypropyl methylcellulose (HPMC) derived polymer.

For the purposes of the present invention, an HPMC derived polymer is an HPMC polymer wherein at least one or more of the “R” groups in a polymer strand is a hydrocarbon moiety other than methyl or hydroxypropyl, for example, phthalate, acetate, and succinate. Moreover, an HPMC derived polymer may include in addition, substitution at the hydroxyl group of a hydroxypropyl moiety, for example, by esterification of the hydroxyl group with a substituent derived from an organic acid, for example, phthalate, acetate or succinate substituent.

It is desirable to have a pharmaceutical composition for oral administration which provides Posaconazole to a patient population with lower patient to patient variability in bioavailability, thus providing consistent pharmacokinetic parameters across a patient population to whom the composition is administered. Moreover, it is desirable to have a composition for oral administration which provides an acceptable plasma level of Posaconazole when administered to a patient in a fasted state. In addition, it is also desirable to expand the scope of the current limited Posaconazole composition processes in order to incorporate a broader range of polymers in the composition.

These needs and other objectives and/or advantages are provided by the present invention, which in one aspect provides a novel composition comprising Posaconazole dissolved or molecularly dispersed in a polymer other than a hydroxypropyl methylcellulose derived polymer.

Moreover, the present invention further provides compositions comprising Posaconazole dispersed in a polymeric matrix prepared using a melt extrusion process in combination with one or more polymeric coatings for a delayed drug release.

Without wanting to be bound by theory it is believed that the inventive compositions display either solid solution morphology, though with a very low degree of long-range ordering, or the compositions of the invention are essentially amorphous. Any and all of these morphologies are contemplated herein by the terms “dissolved in”, “molecularly dispersed in”, “molecular dispersion”, “molten dispersion”, and “dispersion”, used herein for convenience to describe the compositions of the invention at various stages of preparation.

The term “delayed release composition”, as referred to herein, is defined to mean an oral pharmaceutical composition which, when administered, releases the active ingredient at a time later than immediately following its administration and encompasses “modified release” and “enteric” compositions.

One embodiment discloses a delayed release composition comprising Posaconazole up to 50% by weight of the composition. Total amount of Posaconazole in the composition, more preferably comprises up to 30% by weight of the composition.

Another embodiment discloses a polymeric matrix based delayed release composition comprising Posaconazole up to 50% by weight of the composition, more preferably up to 30% by weight of the composition.

The composition of the present invention may further comprise other pharmaceutically active agents suitable for use in combination with Posaconazole for the prevention and treatment of fungal infections and related diseases. Exemplary active agents that can be co-formulated or co-administered with the Posaconazole compositions of the invention include, but are not limited to, steroids, antibiotics, and antifungal agents.

Exemplary antifungal agents include, but are not limited to clotrimazole, fluconazole, ketoconazole, nystatin, itraconazole, amphotericin B, butoconazole nitrate, griseofulvin, ciclopiroz olamine, miconazole nitrate, oxiconazole nitrate, and econazole nitrate.

Another embodiment discloses a delayed release composition comprising Posaconazole, wherein the composition is in the form of a tablet, a capsule, a caplet, beads or granules.

The one or more matrix forming polymers of the present invention may be selected from, but are not limited to one or more of polyvinyl pyrrolidone, polyvinyl acetate-polyvinyl pyrrolidone polymers, acrylic polymers such as polyacrylates, polymethacrylates and copolymers thereof including copolymers based on ethyl acrylate and methyl methacrylate copolymers and methyl methacrylate-methacrylic acid copolymers, polyvinyl alcohol-polyethylene glycol copolymers, polyvinyl acetate phthalate and the like.

Another embodiment discloses a delayed release composition comprising Posaconazole, wherein the composition comprises one or more matrix forming polymers from about 1% to about 40% by weight of the composition, more preferably from about 5% to about 25% by weight of the composition, even more preferably from about 5% to about 20% by weight of the composition.

In some embodiments it is preferred to employ a polymer or polymers in a composition of the invention that form a Posaconazole/polymer composition which has a melting point that is below the point of thermal decomposition of Posaconazole. In some embodiments it is preferred to select a polymer or mixture of polymers for the composition that exhibit poor solubility in an aqueous environment having a pH-value which is more acidic than a value of about pH 2.0, and exhibits good solubility in an aqueous environment which is less acidic than a pH value of from about 6.4 to about 6.8, preferably about pH 6.8.

Suitable enteric coating material may be selected from, but are not limited to one or more of ethyl cellulose, other water-insoluble cellulose derivatives, polymethacrylates and copolymers thereof including copolymers based on ethyl acrylate and methyl methacrylate and methyl methacrylate-methacrylic acid copolymers, polyvinyl acetate phthalate, hydroxypropyl methylcellulose acetate, hydroxypropyl methylcellulose phthalate, cellulose acetate succinate, succinate sodium alginate and stearic acid, fatty acids, waxes and shellac and the like. The enteric coating material may comprise from about 0.1% to about 15% by weight of the composition, more preferably from about 1% to about 10% by weight of the composition.

Suitable non-enteric coating materials may be selected from, but are not limited to cellulose polymers including hydroxypropyl methylcellulose, hydroxypropyl cellulose, vinyl polymers including polyvinyl alcohol, glycols including polyethylene glycol, acrylic polymers and co-polymers thereof and carbohydrates including polydextrose, maltodextrins and the like. The non-enteric coating material may comprise from about 0.1% to about 15% by weight of the composition, more preferably from about 1% to about 10% by weight of the composition.

The pharmaceutical composition may further include one or more pharmaceutically acceptable excipients. The pharmaceutically acceptable excipients may include one or more fillers, disintegrants, binders, wetting agents, lubricants, glidants and the like.

Suitable fillers may include one or more of dextrose, sucrose, maltose, and lactose, sugar-alcohols, which include mannitol, sorbitol, maltitol, xylitol, starch hydrolysates, which include dextrins, and maltodextrins, microcrystalline cellulose, other cellulosic derivatives, dicalcium phosphate, tricalcium phosphate, mixtures thereof and the like.

Suitable disintegrants may include one or more of starch or modified starches, particularly sodium starch glycolate, corn starch, potato starch or pre-gelatinated starch, clays, particularly bentonite, montmorillonite or veegum; celluloses, particularly microcrystalline cellulose like L-hydroxypropylcellulose or carboxymethylcellulose; alginates, particularly sodium alginate or alginic acid; crosslinked celluloses, particularly croscarmellose sodium; gums, particularly guar gum or xanthan gum; crosslinked polymers, particularly crospovidone and the like.

Suitable binders may include one or more of starch, microcrystalline cellulose, highly dispersed silica, mannitol, lactose, polyethylene glycol, polyvinylpyrrol idone, crosslinked polyvinylpyrrol idone, polymethacrylic acid derivatives, ethyl cellulose, methyl cellulose, hydroxyethyl cellulose cross-linked carboxymethylcellulose, hydroxypropylcellulose, hydroxypropyl methylcellulose, natural and synthetic gums, carbomers, dextrin, shellac, zein, gelatin, pregelatinized starch, sodium alginate, synthetic resins and the like. The binder may be present in the composition in an amount of from about 0.1% to about 15% by weight of the composition.

Suitable wetting agent may include one or more of anionic, cationic, nonionic, amphoteric, zwitterionic surfactants or mixtures thereof. Anionic surfactants may include sodium lauryl sulfate, sodium dodecanesulfonate, sodium oleyl sulfate, and sodium laurate mixed with stearates and talc. Cationic surfactants may include benzalkonium chlorides and alkyltrimethylammonium bromides. Nonionic surfactants suitable for use in the compositions described herein may be selected from, for example, polysorbates, poloxamers, polyoxyethylene castor oil derivatives, bile salts, lecithin, 12-Hydroxystearic acid-polyethylene glycol copolymer, and the like.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin and the like.

Another aspect of the present invention is a method for preparing a composition comprising Posaconazole molecularly dispersed in or dissolved in a polymer other than a hydroxypropyl methylcellulose derivative polymer using hot melt extrusion technique.

The ordinarily skilled artisan will recognize that hot melt extrusion involves the conversion of blends from a powder or a granular mix into a product of uniform shape. During this process, polymers are melted with active ingredients and formed into products of different shapes and sizes by forcing polymeric components and active substances including any additives or plasticisers through an orifice or die under controlled temperature, pressure, feeding rate, and screw speed.

In another embodiment, the method includes a process for providing a delayed release Posaconazole composition, wherein the process involves dry mixing Posaconazole and a polymer other than a hydroxypropyl methylcellulose derivative polymer followed by hot melt extruding and co-milling the extrudate to obtain desired granule size. The granules may be mixed with other pharmaceutically acceptable excipients, lubricated and compressed. The compressed tablet may further be coated with an enteric polymer.

In another embodiment, the method includes a process for providing a delayed release Posaconazole composition, wherein the process involves

a) dry mixing Posaconazole and a polymer other than a hydroxypropyl methylcellulose derivative polymer to provide an admixture,
b) forming a molten dispersion by heating the admixture below the melting point of Posaconazole (below about 169° C.) to prevent any degradation of the active ingredient,
c) optionally forming a shaped mass from the dispersion either before or simultaneously with cooling step,
d) co-milling the extrudate to obtain desired granule size,
e) blending the granules with one or more pharmaceutically acceptable excipients,
f) compressing the blend into tablets and optionally coating the tablets with an enteric/non-enteric polymer.

For the purposes of the present invention, the coating step of the process can be carried out by using spraying techniques known in the art or compression coating.

The term “coat” as used herein is defined to mean a coating substantially surrounding a core which provides desirable properties to the dosage form. As is clear to the person of skill in the art, the coat can serve several purposes, including but not limited to protecting the dosage form from environmental conditions, such as light or moisture, providing esthetic or taste-masking properties to the dosage form, making the dosage form easier to swallow or to handle during the production process, or modifying the release properties of the dosage form, such that pharmaceutically active ingredient is released at a different rate from the coated core than from the uncoated core. A coat can itself comprise one or more pharmaceutically active ingredients. One or more than one coat, with the same or different functions or properties, can be applied to a core. The term “coat” includes, but is not limited to, modified release coats, delayed release coats, immediate release coats and non-functional soluble coats.

The release of Posaconazole from the compositions of the present invention may be measured by placing the composition to be tested in a medium in an appropriate dissolution bath. Aliquots of the medium, collected at pre-set intervals, are then injected into a chromatographic system fitted with an appropriate detector to quantify the amount of drug released during the testing intervals.

Another embodiment discloses a delayed release composition comprising Posaconazole, wherein the composition retains at least about 80% of the potency of Posaconazole in the pharmaceutical composition after storing the composition at 40° C. and 75% relative humidity for at least three months.

The oral administration of compositions comprising a composition of the present invention provide improvements in Posaconazole plasma levels and Posaconazole exposure in comparison to other dosage forms whether administered to subjects under fasted conditions or fed conditions, with less variability in observed pharmacokinetic values among a patient population to whom it is administered. Moreover, the food effect seen with other orally administered Posaconazole-containing compositions is substantially eliminated using compositions of the present invention.

Another embodiment discloses a method for preventing and/or treating a fungal infection or related disease comprising administering the Posaconazole composition of the invention to a patient in need of such treatment.

The invention is further illustrated by the following examples which is provided to be exemplary of the invention and does not limit the scope of the invention. While the present invention has been described in terms of its specific embodiments, certain modifications and equivalents will be apparent to those skilled in the art and are intended to be included within the scope of the present invention.

### Example 1

| | | |
|---|---|---|
| Sr. No. | Ingredient | % w/w |

Drug Dispersion

| | | |
|---|---|---|
| 1 | Posaconazole | 25.00 |
| 2 | Eudragit L100-55 | 13.50 |

Intra-Granular Part

| | | |
|---|---|---|
| 3 | Microcrystalline cellulose | 56.00 |
| 4 | Croscarmellose sodium | 1.50 |

Lubrication

| | | |
|---|---|---|
| 5 | Magnesium stearate | 1.50 |

Film Coating

| | | |
|---|---|---|
| 6 | Opadry yellow | 2.50 |

| | |
|---|---|
| Total | 100.00 |

### Process:

Posaconazole and Eudragit L100-55 (anionic copolymer based on methacrylic acid and ethyl acrylate) were mixed uniformly and transferred for hot melt extrusion to form solid dispersion. The extrudates were sized through a co-mill to obtain granules. The granules were blended with microcrystalline cellulose and croscarmellose sodium. The blend was lubricated using magnesium stearate and compressed into tablets using suitable tooling. The tablets obtained were coated with a solution of Opadry Yellow (HPMC based coating system).

### Example 2

| | | |
|---|---|---|
| Sr. No. | Ingredient | % w/w |

Drug Dispersion

| | | |
|---|---|---|
| 1 | Posaconazole | 25.00 |
| 2 | Polyvinyl pyrrolidone | 14 |

Intra-Granular Part

| | | |
|---|---|---|
| 3 | Lactose monohydrate | 55.00 |
| 4 | Crospovidone | 1.50 |

Lubrication

| | | |
|---|---|---|
| 5 | Magnesium stearate | 1.50 |

Film Coating

| | | |
|---|---|---|
| 6 | HPMC phthalate | 3 |

| | |
|---|---|
| Total | 100.00 |

### Process:

Posaconazole and polyvinyl pyrrolidone were mixed uniformly and transferred for hot melt extrusion to form a solid dispersion. The extrudates were sized through a co-mill to obtain granules. The granules were blended with lactose monohydrate and crospovidone. The blend was lubricated using magnesium stearate and compressed into tablets using suitable tooling. The tablets obtained were coated with a solution of HPMC phthalate.

1. A delayed release composition comprising Posaconazole dissolved or molecularly dispersed in a polymer other than a hydroxypropyl methylcellulose derived polymer; wherein the composition is prepared by hot melt extruding an admixture of Posaconazole and the polymer.

2. The delayed release composition according to claim 1, wherein the composition comprises from about 100 mg to about 400 mg Posaconazole.

3. The delayed release composition according to claim 1, wherein the composition comprises Posaconazole up to 30% by weight of the composition.

4. The delayed release composition according to claim 1, wherein the polymer comprises one or more of polyvinyl pyrrolidone, polyvinyl acetate-polyvinyl pyrrolidone polymers, polyvinyl alcohol-polyethylene glycol copolymers or acrylic polymers such as polyacrylates, polymethacrylates and copolymers thereof including copolymers based on ethyl acrylate and methyl methacrylate.

5. The delayed release composition according to claim 1, wherein the composition comprises the polymer from about 5% to about 20% by weight of the composition.

6. The delayed release composition according to claim 1 further comprising one or more pharmaceutically acceptable excipients comprising fillers, disintegrants, binders, wetting agents, lubricants, glidants and the like.

7. The delayed release composition according to claim 1, wherein the composition is in the form of a tablet, a capsule, a caplet, beads or granules.

8. The delayed release composition according to claim 1, wherein the composition does not produce significantly different absorption levels when administered under the fed conditions as compared to the fasting conditions.

9. The delayed release composition according to claim 1, wherein the composition is prepared by process comprising the steps of:
a) blending Posaconazole with the polymer other than a hydroxypropyl methylcellulose derived polymer and one or more pharmaceutically acceptable excipients to form an admixture,
b) heating the admixture to form a melt,
c) extruding the melt into a shaped mass followed by or simultaneously cooling the extrudate,
d) sizing the extrudate through a co-mill to achieve desired granules,
e) blending the granules with one or more pharmaceutically acceptable excipients followed by lubrication,
f) compressing the lubricated blend into tablets, and optionally;
g) coating the tablets.

10. The delayed release composition of claim 1, wherein the composition retains at least about 80% of the potency of Posaconazole in the pharmaceutical composition after storing the composition at 40° C. and 75% relative humidity for at least three months.
